(19) United States
(12) Patent Application Publication
(10) Pub. No.: US 2025/0261847 A1
(43) Pub. Date: Aug. 21, 2025

(54) METHOD AND SYSTEM TO MEASURE OBJECTIVE VISUAL PHOTOSENSITIVITY DISCOMFORT THRESHOLD

(71) Applicant: UNIVERSITY OF MIAMI, Miami, FL (US)

(72) Inventors: Yu-Cherng Channing CHANG, Miami, FL (US); Marco RUGGERI, Miami, FL (US); Alex GONZALEZ, Miami, FL (US); Mariela C. AGUILAR, Miami, FL (US); Fabrice MANNS, Miami, FL (US); Jean-Marie PAREL, Miami, FL (US)

(21) Appl. No.: 18/867,355

(22) PCT Filed: May 22, 2023
(86) PCT No.: PCT/US2023/023130

§ 371 Date: Nov. 19, 2024

Related U.S. Application Data
(60) Provisional application No. 63/344,366, filed on May 20, 2022.

(51) Int. Cl. A61B 3/11; A61B 3/00; A61B 3/06; A61B 3/14; G06T 7/00; G06V 10/82; G06V 40/16

(52) U.S. Cl. CPC A61B 3/112; A61B 3/0025; A61B 3/063; A61B 3/14; G06T 7/0012; G06T 2207/10016; G06T 2207/20081; G06T 2207/20084; G06T 2207/30041; G06T 2207/30201; G06V 10/82; G06V 40/174

(57) ABSTRACT

Method to measure a visual photosensitivity discomfort threshold or presence of a condition associated therewith within a subject, includes obtaining, by one or more processors, a plurality of images of the subject captured while the at least one pupil and corresponding palpebral fissure contour was being subjected to a light stimuli; determining, by the one or more processors, executing instructions for a neural network having been trained using a plurality of images of a plurality of subjects captured at a plurality of different illumination levels, an output value corresponding to a measure of visual photosensitivity discomfort threshold in which the visual photosensitivity discomfort threshold is defined by an estimated illuminance of the retina of the subject, and outputting, by the one or more processors, the output value in a report.

### CROSS-REFERENCE TO RELATED APPLICATIONS

This PCT application claims priority to, and benefit under 35 U.S.C. § 119 (e) of, U.S. Provisional Patent Application No. 63/344,366 filed May 20, 2022, which is hereby incorporated by reference herein in its entirety.

### BACKGROUND

In individuals with photosensitivity, exposure to certain types or intensities of light can trigger abnormal responses.

Photosensitivity analysis can be used for the diagnosis and treatment of ophthalmological conditions. Photosensitivity analysis can include determining a subject's visual photosensitivity threshold, which can be a measure of what light intensity causes discomfort in a subject. Photosensitivity analysis can be used to predict and prevent various conditions that affect the photosensitivity thresholds of a subject, for example, photophobia, migraines, cataracts, and retinal disorders. Improvements to photosensitivity analysis can improve the treatment, prevention, and diagnosis of disorders related to photosensitivity. Additionally, improvements to photosensitivity analysis can be used to improve protective eyewear, improve lighting designs for artificial light, and perform therapies using light.

There is a benefit to improving photosensitivity analysis.

### SUMMARY

An exemplary system and method are disclosed to provide an objective measure of Visual Photosensitivity Discomfort Threshold (VPT, also referred to herein as OVPT) based on retinal illuminance. In some embodiments, to evaluate ocular photosensitivity based on retinal illuminance, a deep learning system and method are configured to evaluate pupil responses as compared to a large population set. The deep learning system and method can be configured to detect facial expressions (e.g., associated with light-induced discomfort) and link them with pupil responses to assess Visual Photosensitivity Discomfort Threshold (VPT) objectively (OVPT). The VPT/OVPT measurement operations may be applied to stimulus illuminance, e.g., to improve the repeatability of measurements. The evaluation based on retinal illuminance provides for a more robust (e.g., more repeatable) measure of ocular photosensitivity and a subject's perception of discomfort in response to light stimuli.

The term “photosensitivity threshold,” or “VPT,” as used herein, refers to the minimum level of light intensity or frequency required to elicit a specific physiological or behavioral response in an individual who is sensitive to light. It is a measure of the sensitivity of the visual system to light. The term refers to a measure that is objectively or subjectively determined.

The term “ocular photosensitivity threshold,” as used herein, can refer to the intensity of light that causes pain or discomfort to the eyes. The term refers to a measure that is objectively determined.

Expressing VPT or OVPT in terms of retinal illuminance may improve measurement repeatability, among other things. Detecting facial expressions may improve measurement repeatability even if the VPT/OVPT is not expressed in terms of retinal illuminance, but in terms of stimulus illuminance, for example. VPT or OVPT expressed in terms of stimulus illuminance may be considered as a conventional method currently used in most systems. Indeed, expressing VPT or OVPT in terms of retina illuminance and facial expression detection are independent methods to improve VPT or OVPT measurement repeatability; they can be used together or not. For example, facial expression detection may improve measurement repeatability even if VPT or OVPT is expressed in terms of stimulus illuminance and if AI is not used to detect pupil size to express VPT or OVPT in terms of retinal illuminance.

In an aspect, a method is disclosed to measure of visual photosensitivity discomfort threshold (VPT/OVPT) or presence of a condition associated therewith within a subject, the method comprising obtaining, by one or more processors, a plurality of images of the subject (e.g., digital video), wherein each image includes a representation of i) at least one pupil and ii) a corresponding palpebral fissure contour, of the subject captured while the at least one pupil and corresponding palpebral fissure contour was being subjected to a light stimuli (e.g., of a known illuminance intensity); determining, by the one or more processors, executing instructions for a neural network having been configured (trained) using a plurality of images of a plurality of subjects captured at a plurality of different illumination levels, an output value corresponding to a measure of visual photosensitivity discomfort threshold in which the visual photosensitivity discomfort threshold is defined by an estimated illuminance of the retina of the subject, and outputting, by the one or more processors, the output value in a report (e.g., digital or printed report; control interface, e.g., for a stimulus illumination tool for VPT/OVPT measurement, etc.) (e.g., wherein the output value is used to diagnose the subject for photophobia or light sensitivity condition; or used to evaluate effectiveness or adjust configuration of eyewear or optical instruments for reducing a subject's reaction or discomfort to light sensitivity causing stimuli).

Examples of neural networks that may be used for the segmentation of the pupil include convolutional neural networks, deep convolutional encoder-decoder, Feature Pyramid Networks, and other deep neural networks such as PSPnet, Segnet, U-Net, FCN, LinkNet, and FPN, among other described herein.

In some embodiments, the method further includes outputting, by the one or more processors, visualization indicating an estimated area of the at least one pupil in at least one image, or a portion thereof, of the plurality of images of the subject, wherein the estimated area was generated by the neural network and used by the neural network to determine the output value.

In some embodiments, the method further includes outputting, by the one or more processors, visualization indicating a palpebral fissure contour in at least one image, or a portion thereof, of the plurality of images of the subject, wherein the palpebral fissure contour was generated by the neural network and used by the neural network to determine the output value.

In some embodiments, the plurality of images are acquired from an ocular photosensitivity analyzer.

In some embodiments, the plurality of images are acquired from a video camera system comprising illumination control.

In some embodiments, the neural network had been trained with a second plurality of images of a second plurality of subjects quantified as having different eye sizes.

In some embodiments, the neural network had been trained with a second plurality of images of the plurality of subjects at different head motions.

In some embodiments, the obtained plurality of images each includes a representation of a facial area of the subject, wherein the neural network has been configured to classify facial expressions selected from the group consisting of blinking, squinting, and frowning, and wherein parameters associated with the classified facial expressions are used to generate the determined output value.

In some embodiments, the neural network employs features extracted from at least one of: (i) a developed eye model, (ii) an optical model of an ocular photosensitivity analyzer or camera system, or (iii) a physical eye model comprising a variable associated with pupil diameter and/or refractive error.

In some embodiments, the obtained plurality of images each includes a representation of a facial area of the subject, wherein a second neural network has been configured to generate a facial expression parameter; the facial expression parameter is used to generate the determined output value.

In some embodiments, the neural network employs a feature extracted from a physical facial model of the subject.

In some embodiments, the neural network comprises an image segmentation algorithm.

In some embodiments, the output value defined by the estimated illuminance of the retina of the subject and as generated by the neural network has greater measurement repeatability as compared to the output value being defined by stimuli illuminance.

In some embodiments, the output value is used to diagnose the subject for photophobia or light sensitivity condition.

In some embodiments, the output value is used to evaluate the effectiveness of eyewear or optical instruments.

In some embodiments, the output value is used to adjust the configuration of eyewear or optical instruments to reduce a subject's reaction or discomfort to light sensitivity-causing stimuli).

In some embodiments, the one or more processors are located in a local computing device or a cloud platform.

In another aspect, a system is disclosed comprising a processor; a memory having instructions stored thereon, wherein the instructions when executed by the processor, cause the processor to measure of visual photosensitivity discomfort threshold (VPT/OVPT) or presence of a condition associated therewith within a subject by: obtaining, a plurality of images of the subject (e.g., digital video), wherein each image includes a representation of i) at least one pupil and ii) a corresponding palpebral fissure contour, of the subject captured while the at least one pupil and corresponding palpebral fissure contour was being subjected to a light stimuli (e.g., of a known illuminance intensity); determining executing instructions for a neural network having been configured (trained) using a plurality of images of a plurality of subjects captured at a plurality of different illumination levels, an output value corresponding to a measure of visual photosensitivity discomfort threshold in which the visual photosensitivity discomfort threshold is defined by an estimated illuminance of the retina of the subject, and outputting the output value in a report (e.g., digital or printed report; control interface, e.g., for a stimulus illumination tool for VPT/OVPT measurement, etc.) (e.g., wherein the output value is used to diagnose the subject for photophobia or light sensitivity condition; or used to evaluate effectiveness or adjust configuration of eyewear or optical instruments for reducing a subject's reaction or discomfort to light sensitivity causing stimuli).

In some embodiments, the system further includes an ocular photosensitivity analyzer, wherein the ocular photosensitivity analyzer comprises at least one hardware processor; a programmable light source comprising a plurality of multi-spectra light modules configured to emit light at a range of wavelengths; a sensing system comprising one or more sensors; and one or more software modules that are configured to, when executed by the at least one hardware processor, receive an indication of a lighting condition comprising one or more wavelengths of light, configure the programmable light source to emit light according to the lighting condition, and, for each of one or more iterations, activate the programmable light source to emit the light according to the lighting condition, and collect a response, by a subject, to the emitted light via the sensing system.

In another aspect, a non-transitory computer readable medium is disclosed having instructions stored thereon, wherein execution for the instructions by the processor, cause the processor to measure of visual photosensitivity discomfort threshold (VPT/OVPT) or presence of a condition associated therewith within a subject by obtaining, a plurality of images of the subject (e.g., digital video), wherein each image includes a representation of i) at least one pupil and ii) a corresponding palpebral fissure contour, of the subject captured while the at least one pupil and corresponding palpebral fissure contour was being subjected to a light stimuli (e.g., of a known illuminance intensity); determining executing instructions for a neural network having been configured (trained) using a plurality of images of a plurality of subjects captured at a plurality of different illumination levels, an output value corresponding to a measure of visual photosensitivity discomfort threshold in which the visual photosensitivity discomfort threshold is defined by an estimated illuminance of the retina of the subject, and outputting the output value in a report (e.g., digital or printed report; control interface, e.g., for a stimulus illumination tool for VPT/OVPT measurement, etc.) (e.g., wherein the output value is used to diagnose the subject for photophobia or light sensitivity condition; or used to evaluate effectiveness or adjust configuration of eyewear or optical instruments for reducing a subject's reaction or discomfort to light sensitivity causing stimuli).

### BRIEF DESCRIPTION OF THE DRAWINGS

The accompanying drawings, which are incorporated in and constitute a part of this specification, illustrate embodiments and, together with the description, serve to explain the principles of the methods and systems. The patent or application file contains at least one drawing executed in color.

FIG. 1 illustrates a flowchart of an exemplary method for measuring the VPT/OVPT of a subject in accordance with an illustrative embodiment.

FIG. 2 illustrates an AI system configured to detect pupil and palpebral fissure contours from the digital video sequences in accordance with an illustrative embodiment.

FIG. 3 shows experimental results and examples of the AI system of FIG. 2 to detect pupil and palpebral fissure contours from the digital video sequences in accordance with an illustrative embodiment.

FIG. 4 illustrates a method of validating and confirming the repeatability between VPT/OVPT measurements in accordance with an illustrative embodiment.

FIG. 5 illustrates a method of assessing whether facial expression recognition improves measurement repeatability.

FIG. 6 illustrates an exemplary computer that can be used to the AI system of FIG. 2 in accordance with an illustrative embodiment.

### DETAILED DESCRIPTION

Each and every feature described herein, and each and every combination of two or more of such features, is included within the scope of the present invention provided that the features included in such a combination are not mutually inconsistent.

It is understood that throughout this specification, the identifiers “first,” “second,” “third,” “fourth,” “fifth,” “sixth,” and such are used solely to aid in distinguishing the various components and steps of the disclosed subject matter and such, are not intended to imply any particular order, sequence, amount, preference, or importance to the components or steps modified by these terms.

Throughout this application, various publications may be referenced. The disclosures of these publications in their entirety are hereby incorporated by reference into this application in order to more fully describe the state of the art to which the methods and systems pertain. Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. For example, “[3]” refers to the 3rd reference in the list. All references cited and discussed in this specification are incorporated herein by reference in their entirety and to the same extent as if each reference was individually incorporated by reference.

As discussed above, systems and methods are disclosed for improving the measurement of the visual photosensitivity discomfort threshold (VPT/OVPT). Embodiments of the present disclosure can create a VPT/OVPT measure based on retinal illuminance. Furthermore, embodiments of the present disclosure include validated and individualized optical models of the eye that consider refractive error and pupil size for accurate estimation of retinal illuminance.

### EXAMPLE SYSTEM

The stimulus illumination tool for VPT/OVPT measurement is, in some embodiments, an automated instrument that can quantify the VPT/OVPT in light sensitive subjects. The VPT/OVPT measurement and stimulus illumination tool may automatically generate light stimuli of varying intensity to determine the VPT/OVPT under illumination conditions emulated by an array of LEDs. An example system is the one descriebd in Aguilar, M. C., Gonzalez, A., Rowaan, C., de Freitas, C., Alawa, K. A., Durkee, H., Feuer, W. J., Manns, F., Asfour, S. S., Lam, B. L. & Parel, J. A. Automated instrument designed to determine visual photosensitivity thresholds. Biomed Opt Express 9, 5583-5596, doi: 10.1364/boe.9.005583 (2018) and US Patent Publciation No. 20220409041, each of which is incorporated by reference herein in its entirety.

An implementation of the VPT/OVPT measurement and stimulus illumination tool may include a programmable light source comprising a plurality of multi-spectra light modules configured to emit light at a range of wavelengths as described in [1], which can be used to assess the effect of spectral filters on VPT/OVPT under different illumination conditions. Obtaining highly repeatable measurements with these instruments facilitates the accurate detection of significant changes in VPT/OVPT across different subjects and/or illumination conditions. However, a substantial number of subjects can show highly variable VPT/OVPT when repeated measurements are performed using the current ocular photosensitivity analyzer testing paradigm.

By combining biometric information, including pupil area and optical models of the eye, embodiments of the present disclosure can express VPT/OVPT in terms of retinal illuminance. Furthermore, embodiments of the present disclosure can combine these parameters with the detection and/or measurement of facial expression parameters to detect physical discomfort associated with measuring VPT/OVPT.

Embodiments of the present disclosure can overcome limitations affecting measurement repeatability. For example, embodiments of the present disclosure can measure retinal illuminance. Retinal illuminance can more directly relate to the photosensitivity response than methods that measure the photosensitivity response in terms of the photometric properties of the light source (i.e., stimulus illuminance). In existing techniques for measuring VPT/OVPT that use a fixed stimulus illuminance, the fixed stimulus may produce different levels of illuminance at the retina depending on the optics of the eye (i.e., refractive error), pupil response, and frequency of blinking and squinting, reducing the repeatability of photosensitivity measurements.

Additionally, some embodiments of the present disclosure can perform an objective analysis of the VPT/OVPT, in contrast to conventional methods in which the VPT is determined by querying the participant on which light stimuli are uncomfortable (making the testing paradigm highly subjective).

Additionally, some embodiments of the present disclosure can include deep learning approaches for robust automated pupillometry, measuring VPT/OVPT based on retinal illuminance, and deep learning approaches to automatically detect facial expressions associated with light-induced discomfort.

### EXAMPLE METHOD

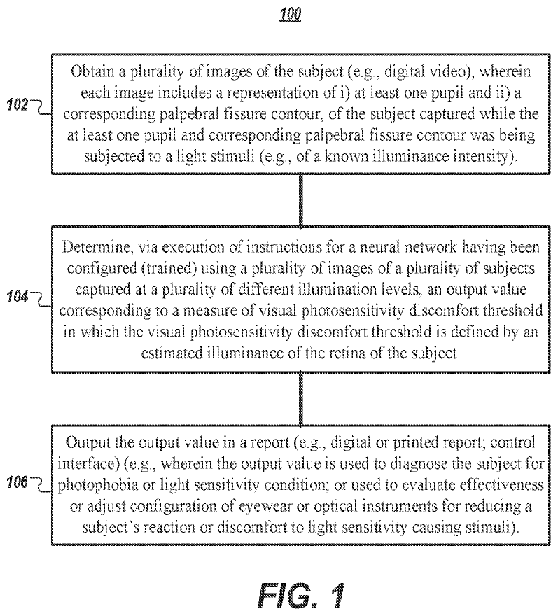

FIG. 1 is a flow diagram of method 100 for measuring visual photosensitivity discomfort threshold (VPT/OVPT) or the presence of a condition associated therewith within a subject. Images of the subject are obtained 102. The images that are obtained 102 can include images of a pupil, a palpebral fissure contour of an eye, and/or the entire face of the subject.

Non-limiting examples of image acquisition (102) include a VPT/OVPT measurement and stimulus illumination tool or a video camera system. In some embodiments, the VPT/OVPT measurement and stimulus illumination tool and/or video camera system include an illumination control.

One or more processors can execute instructions for executing a neural network (NN). The NN, in some embodiments (e.g., CNN or other DNN as described herein), can be trained using one or more images of one or more test subjects. One or more images can include images of the face and/or pupils of the subjects. Additionally, the images of one or more subjects can be captured at different levels of illumination. Based on the images obtained 102, the NN can determine (104) an output value corresponding to a measure of VPT/OVPT that is based on the estimated illuminance of the retina of the subject.

The NN can include an image segmentation algorithm, and a non-limiting example of a suitable image segmentation algorithm is a pyramid scene parsing network (PSPNet), or other convolutional neural network, deep convolutional encoder-decoder, Feature Pyramid Network, and deep neural network such as Segnet, U-Net, FCN, LinkNet, and FPN.

In some embodiments of the present disclosure, the NN is trained with images that are captured using different parameters than the image of the subject. For example, the NN can be trained us using images with different eye sizes than the subject or different head motions than the subject.

The neural network can be trained to develop a model of a feature and extract that model from the subject images. Non-limiting examples of features that can be extracted from the training images include a developed eye model, an optical model of an ocular photosensitivity analyzer or camera system, and/or a physical eye model. The neural network may include multiple types of networks., e.g., having a convolutional neural network that is combined with or be part of another neural network such as a recurrent neural network (RNN) or any other types of networks described herein.

In some embodiments of the present disclosure, more than one NNs are trained to determine (104) the output value. For example, a second NN can be used to generate a facial expression parameter that can be used to generate an output value, and the second NN can generate a facial expression parameter that can be used to determine 104 as an output value.

One or more processors can generate (106) the output value in a report. The report may include the output value can be used to evaluate the effectiveness of eyewear or optical instruments. The output value can be defined as the estimated illuminance of the retina of the subject. Non-limiting examples of how the output value can be used include estimating the effectiveness of eyewear and optical instruments, adjusting eyewear or optical instruments, and/or diagnosing a subject with photophobia or other light sensitivity conditions.

It is contemplated that other values or images may be output 106 by one or more processors. In some embodiments of the present disclosure, the output includes a visualization that indicates an estimated area of a pupil in an image of the subject. The estimated area of the pupil can be determined by the NN to generate an output value (e.g., an output value representing VPT/OVPT for the subject). Additionally, in some embodiments of the present disclosure, the output 106 can include a visualization generated by the NN indicating the palpebral fissure contour of the subject.

In addition to neural networks, other machine learning, and AI methods may be employed.

Machine Learning. In addition to the machine learning features described above, the system can be implemented using one or more artificial intelligence and machine learning operations. The term “artificial intelligence” can include any technique that enables one or more computing devices or comping systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target) during training with a labeled data set (or dataset). In an unsupervised learning model, the algorithm discovers patterns among data. In a semi-supervised model, the model learns a function that maps an input (also known as a feature or features) to an output (also known as a target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as an input layer, an output layer, and optionally one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., an error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similar to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., error such as L1 or L2 loss), during training. This disclosure contemplates that any algorithm that finds the minimum of the cost function can be used. LR classifiers are known in the art and are therefore not described in further detail herein.

An Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is an unsupervised classification model that classifies new data points based on similarity measures (e.g., distance functions). The k-NN classifiers are trained with a data set (also referred to herein as a “dataset”) to maximize or minimize a measure of the k-NN classifier's performance during training. This disclosure contemplates any algorithm that finds the maximum or minimum. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

Example Automated Pupillometry using Deep Learning

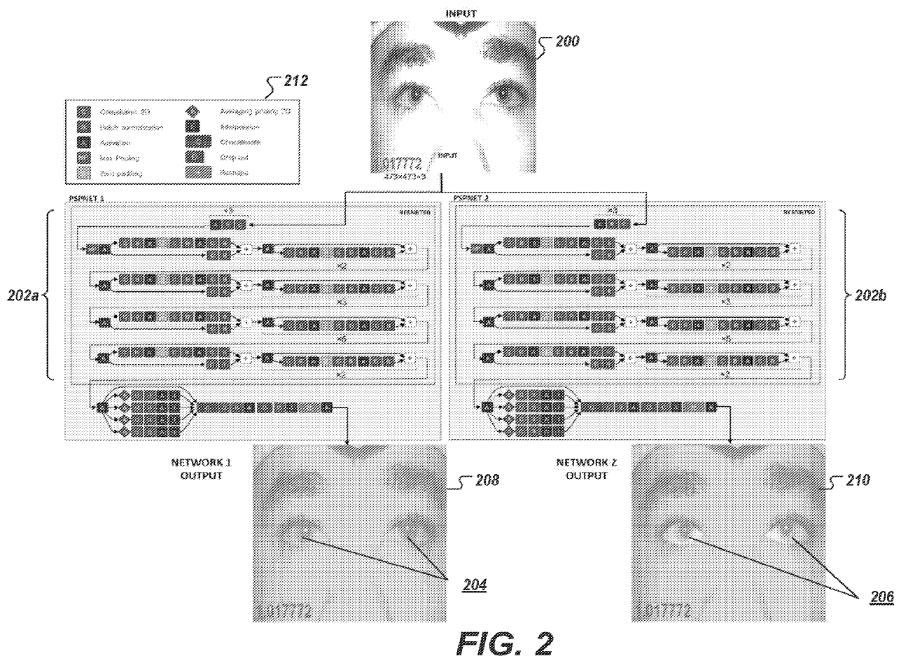

FIG. 2 shows two examples of neural networks 200 (shown as 200a and 200b) based on RESNET and PSPNet that can be employed to provide a robust platform for the detection of the pupil and palpebral fissure contours from the digital video sequences 202 acquired with the VPT/OVPT measurement and stimulus illumination tool, e.g., described in [1]. The highlighted shades (204, 206) in the output images (208, 210) represent the area of the pupil (204) and the palpebral fissure (206) determined by the networks. Components 212 of the neural networks (200a, 200b) include the convolution layer (2D), batch nominalization, activation layer, max pooling layer, zero padding layer, average pooling layer (2D), interpolation layer, concatenate layer, drop-out layer, and reshape layer.

The neural networks can perform repeatable photosensitivity measurements by expressing photosensitivity in terms of retinal illuminance and/or by incorporating automated detection of facial expressions associated with light discomfort into the testing paradigm. Deep learning approaches can enhance the reliability of photosensitivity measurements acquired with the VPT/OVPT measurement and stimulus illumination tool.

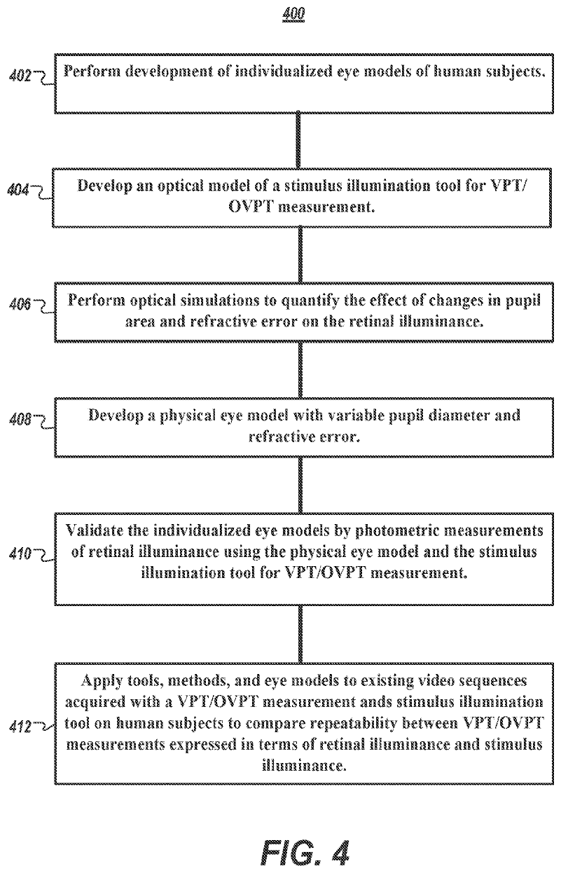

FIG. 4 shows an example method for developing (402) individualized eye models of human subjects. Method 400 can include developing (404) an optical model of a stimulus illumination tool for VPT/OVPT measurement. The deep learning-based algorithms can also leverage the information provided by digital video sequences of the eyes and face acquired with the VPT/OVPT measurement and stimulus illumination tool to automatically and reliably measure pupil area and recognize facial expressions associated with light discomfort (e.g., blinking, squinting, frowning). Measurements of pupil area can be used in combination with individualized optical models of the eye to express light stimuli in terms of retinal illuminance, providing photosensitivity measurements that may reflect more accurately the neurophysiological response to light exposure. In some embodiments of the present disclosure, methods to automatically recognize and track facial expressions associated with light-induced discomfort can be implemented in the testing paradigm to improve measurement repeatability.

Method 400 can include performing (406) optical simulations to quantify the effect of changes in pupil area and refractive error on the retinal illuminance. The quantification may be based on the neural networks 200 described above or, in alternative embodiments, the template-matching technique described in [1]. Software developed for the detection of the pupil and palpebral fissure contours in the VPT/OVPT measurement and stimulus illumination tool that is based on a template-matching technique [1] can be highly sensitive to ocular reflections and image distortions generated by eye and head motion. As a result, pupil and palpebral fissure contours can be inaccurately determined in a significant number of frames, leading to measurement errors.

Training on subjects and refinement of the model can be used to generalize segmentation accuracy across all subjects and all conditions (e.g., large eye and head motion). In some embodiments of the present disclosure, the NN analyzes video produced from an onboard camera that is part of the VPT/OVPT measurement and stimulus illumination tool.

Method 400 can include developing (408) a physical eye model. The eye model may be defined by variables including pupil diameter and refractive error. The eye model determined by the neural network-based image processing tool can be configured to detect pupil and palpebral fissure contours from the digital videos acquired with a VPT/OVPT measurement and stimulus illumination tool. The neural network-based image processing tool can be trained using existing human data acquired with the VPT/OVPT measurement and stimulus illumination tool.

Method 400 can include validating (410) the individualized eye models by photometric measurements of retinal illuminance. The validation may use the physical eye model and the stimulus illumination tool for VPT/OVPT measurement. The validation can use a cohort of test subjects, e.g., by a cross-validation technique. Method 400 can include applying (412) the tools, methods and eye models to existing video sequences acquired with a VPT/OVPT measurement and stimulus illumination tool on human subjects to compare repeatability between VPT/OVPT measurements expressed in terms of retinal illuminance and stimulus illuminance.

Example Measure of Visual Photosensitivity Discomfort Threshold based on Retinal Illuminance and Automated Recognition of Facial Expressions associated with Light-Induced Discomfort

A VPT/OVPT measurement and stimulus illumination tool can measure VPT/OVPT in terms of the photometric properties of the light source (i.e., stimulus illuminance). Embodiments of the present disclosure include eye models that include optical simulations and photometric measurements. These can enable expressing VPT/OVPT in terms of retinal illuminance, a parameter that may more directly relate to the photosensitivity response. FIG. 2 shows an example method that can be used in conjunction with the other methods described herein.

Visual photosensitivity discomfort thresholds are currently determined by querying (via verbal inquiry) the participant on which light stimuli intensity are uncomfortable. This approach makes the testing paradigm highly subjective. Facial expression changes, and certain expressions (e.g., blinking rate, squinting, frowning) are known indicators/behaviors of discomfort and pain [3-7].

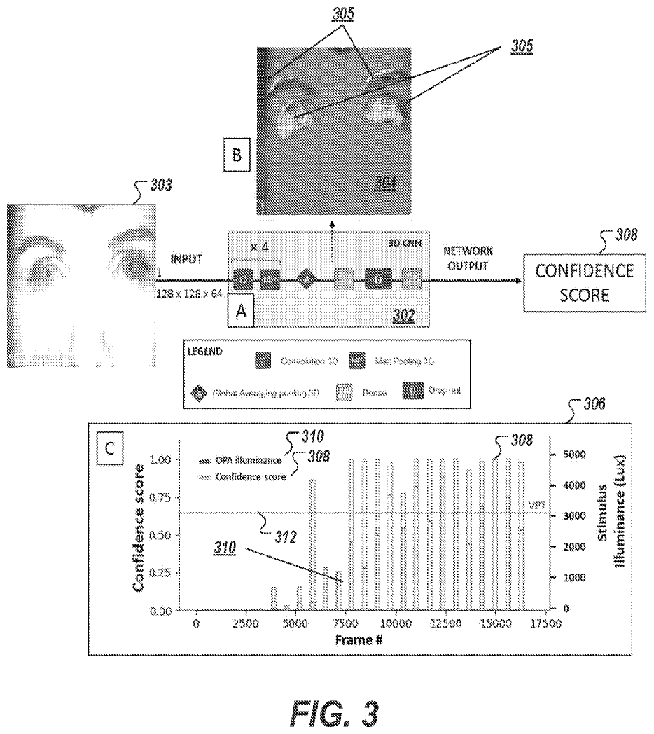

FIG. 3 shows an example 3D CNN 302 that can be employed to recognize or classify facial expressions associated with light-induced discomfort from the digital videos 303 acquired with the onboard camera of the VPT/OVPT measurement and stimulus illumination tool. In the example shown in FIG. 3, the 3D CNN 302 includes 4 sets of convolution layers (3D) and max pooling layers. The max pooling layers then connect to the averaging pooling layer (3D) that connects to a dense, deep neural network. The neural network then connects to a dropout that is then connected to a second dense neural network.

The convolutional neural network 302 is configured to detect light-induced facial expressions to generate an intermediate output 304. In the example shown in FIG. 3, the network activations provide overlay over the image in which areas of interest 305 related to signs of discomfort (e.g., blinking, squinting) are highlighted.

Graph 306 shows the confidence score 308 generated by the 3D CNN 302. Graph 306 also shows the stimulus illuminance (310) recorded during an experiment using a VPT/OVPT measurement and stimulus illumination tool. Graph 306 also shows the determined VPT/OVPT 312 on which the highlight 305 is based. The VPT/OVPT are determined using the standard testing paradigm described in [1]. In the example, testing was performed on a subject wearing spectral filters to confirm the ability of the NN to detect activation areas even when a spectacle frame is used.

Deep learning approaches can evaluate the relationship between facial expressions (and changes herein) and light-intensity discomfort. These approaches can be used to evaluate VPT/OVPT measurement variability, which may be correlated with the subjective testing paradigm, and the defensive facial expressions of squinting and frowning. Other classes of networks can be used to assess discomfort and are collectively referred to as “convRNN” herein.

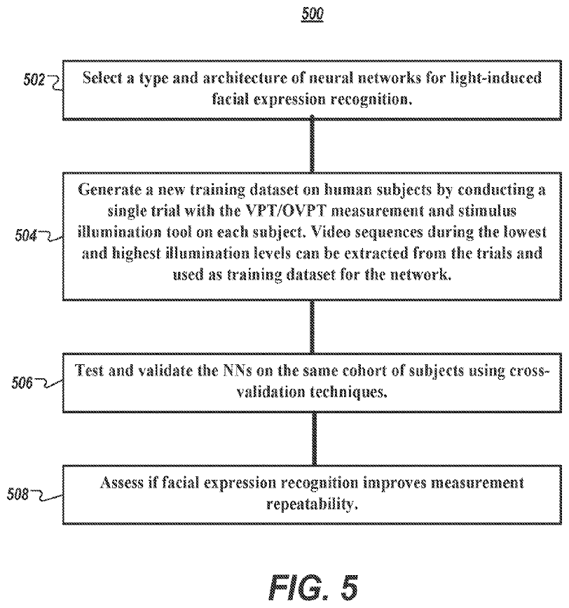

FIG. 5 shows an example method 500 o recognize or classify facial expressions associated with light-induced discomfort. Method 500 can include selecting (502) a type and architecture of neural networks for light-induced facial expression recognition.

Method 500 can include generating (504) a new training dataset on human subjects by conducting a single trial with the VPT/OVPT measurement and stimulus illumination tool on each subject. Video sequences during the lowest and highest illumination levels can be extracted from the trials and used as a training dataset for the network.

A model can be trained on video sequences from a few subjects during the lowest and highest illumination levels provided by the VPT/OVPT measurement and stimulus illumination tool in that trial, which were respectively classified as no light-induced discomfort and light-induced discomfort.

In the example shown in FIG. 3, the NN was trained to learn to focus on activity (see image 304) in the eyebrows and eyes/eyelid, which are involved in discomfort-related expressions (e.g., blinking and squinting).

Method 500 can include testing (506) and validating the NNs on the same cohort of subjects using cross-validation techniques. In the example, a classification experiment was performed with the trained NN on a full video sequence of a participant while the VPT/OVPT measurement and stimulus illumination tool generated light stimuli of varying intensities (FIG. 3). The NN assigned a confidence score of discomfort to video sequences from each stimulus level (FIG. 3), indicating the probability of light-induced discomfort. The confidence score ranged between “0” and “1,” in which “1” indicates a high probability of discomfort and “0” indicates a low probability of discomfort. The confidence score can be used as a metric to test the subjects' reactions to the individual stimuli. Therefore, the confidence score can be developed as a potential covariate in the testing paradigm to reduce subjective bias variability, potentially improving measurement repeatability.

Method 500 can include assessing (508) if facial expression recognition improves measurement repeatability. The digital videos of the subjects that have shown low repeatable behavior can be processed with the NN. For these subjects, VPT/OVPT can be recalculated using the confidence score as a covariate. In some embodiments, the confidence score threshold can be determined by applying the NN to the data of the participants that have shown highly repeatable behavior. Measurement repeatability of VPT/OVPT with and without using confidence scores can be assessed and compared.

The methods of detecting facial expressions associated with light-induced discomfort described herein can be combined with other VPT/OVPT measurements, including the VPT/OVPT measurement methods, e.g., to improve the accuracy and/or repeatability of the VPT/OVPT measurements.

### Discussion

Expressing VPT in terms of retinal illuminance may improve measurement repeatability. However, detecting facial expressions might improve measurement repeatability even if the VPT is not expressed in terms of retinal illuminance, but in terms of stimulus illuminance, for example. VPT expressed in terms of stimulus illuminance is the conventional method used in our systems now.

Expressing VPT in terms of retina illuminance and facial expression detection are independent methods to improve VPT measurement repeatability; they can be used together or not. For example, facial expression detection may improve measurement repeatability even if VPT is expressed in terms of stimulus illuminance, and if AI was not used to detect pupil size to express VPT in terms of retinal illuminance. Other ocular photosensitivity evaluations (e.g., template matching techniques [1]) may be employed that assess ocular photosensitivity based on retinal illuminance that does not necessarily rely on deep neural networks.

### Example Computing Device

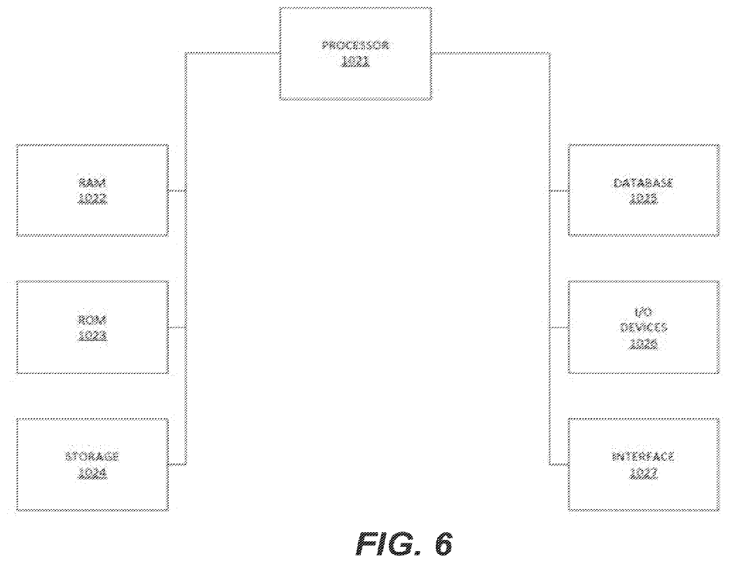

FIG. 6 illustrates an exemplary computer that may comprise all or a portion of an automated design tool for determining the VPT/OVPT of a subject or training a NN for determining the VPT/OVPT of a subject. Conversely, any portion or portions of the computer illustrated in FIG. 6 may comprise all or part of the system for determining the VPT/OVPT of a subject or training a NN for determining the VPT/OVPT of a subject. As used herein, “computer” may include a plurality of computers. The computers may include one or more hardware components such as, for example, a processor 1021, a random-access memory (RAM) module 1022, a read-only memory (ROM) module 1023, a storage 1024, a database 1025, one or more input/output (I/O) devices 1026, and an interface 1027. Alternatively, and/or additionally, the computer may include one or more software components such as, for example, a computer-readable medium including computer-executable instructions for performing a method associated with the exemplary embodiments such as, for example, an algorithm for determining a property profile gradient. It is contemplated that one or more of the hardware components listed above may be implemented using the software. For example, storage 1024 may include a software partition associated with one or more other hardware components. It is understood that the components listed above are exemplary only and not intended to be limiting.

Processor 1021 may include one or more processors, each configured to execute instructions and process data to perform one or more functions associated with a computer for controlling a system (e.g., automated design tool) and/or receiving and/or processing and/or transmitting data associated with electrical sensors. Processor 1021 may be communicatively coupled to RAM 1022, ROM 1023, storage 1024, database 1025, I/O devices 1026, and interface 1027. Processor 1021 may be configured to execute sequences of computer program instructions to perform various processes. The computer program instructions may be loaded into RAM 1022 for execution by processor 1021.

RAM 1022 and ROM 1023 may each include one or more devices for storing information associated with the operation of processor 1021. For example, ROM 1023 may include a memory device configured to access and store information associated with the computer, including information for identifying, initializing, and monitoring the operation of one or more components and subsystems. RAM 1022 may include a memory device for storing data associated with one or more operations of processor 1021. For example, ROM 1023 may load instructions into RAM 1022 for execution by processor 1021.

Storage 1024 may include any type of mass storage device configured to store information that processor 1021 may need to perform processes consistent with the disclosed embodiments. For example, storage 1024 may include one or more magnetic and/or optical disk devices, such as hard drives, CD-ROMs, DVD-ROMs, or any other type of mass media device.

Database 1025 may include one or more software and/or hardware components that cooperate to store, organize, sort, filter, and/or arrange data used by the computer and/or processor 1021. For example, database 1025 may store data related to the plurality of thrust coefficients. The database may also contain data and instructions associated with computer-executable instructions for controlling a system (e.g., a multi-material printer) and/or receiving and/or processing and/or transmitting data associated with a network of sensor nodes used to measure water quality. It is contemplated that database 1025 may store additional and/or different information than that listed above.

I/O devices 1026 may include one or more components configured to communicate information with a user associated with a computer. For example, I/O devices may include a console with an integrated keyboard and mouse to allow a user to maintain a database of digital images, results of the analysis of the digital images, metrics, and the like. I/O devices 1026 may also include a display, including a graphical user interface (GUI) for outputting information on a monitor. I/O devices 1026 may also include peripheral devices such as, for example, a printer, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 1027 may include one or more components configured to transmit and receive data via a communication network, such as the Internet, a local area network, a workstation peer-to-peer network, a direct link network, a wireless network, or any other suitable communication platform. For example, interface 1027 may include one or more modulators, demodulators, multiplexers, demultiplexers, network communication devices, wireless devices, antennas, modems, radios, receivers, transmitters, transceivers, and any other type of device configured to enable data communication via a wired or wireless communication network.

The figures illustrate the architecture, functionality, and operation of possible implementations of systems, methods, and computer program products according to various implementations of the present invention. In this regard, each block of a flowchart or block diagram may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

Any combination of one or more computer-readable medium(s) may be used to implement the systems and methods described hereinabove. The computer-readable medium may be a computer-readable signal medium or a computer-readable storage medium. A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

Program code embodied on a computer-readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to the arrangement of steps or operational flow, plain meaning derived from grammatical organization or punctuation, the number or type of embodiments described in the specification.

One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language if desired. In any case, the language may be a compiled or interpreted language, and it may be combined with hardware implementations.

### Conclusion

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

### Conclusion

Various sizes and dimensions provided herein are merely examples. Other dimensions may be employed.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications, and publications, as listed below and throughout this document, describes various application and systems that could be used in combination the exemplary system and are hereby incorporated by reference in their entirety herein.

### REFERENCES

[1] Aguilar, M. C., Gonzalez, A., Rowaan, C., de Freitas, C., Alawa, K. A., Durkee, H., Feuer, W. J., Manns, F., Asfour, S. S., Lam, B. L. & Parel, J. A. Automated instrument designed to determine visual photosensitivity thresholds. Biomed Opt Express 9, 5583-5596, doi: 10.1364/boe.9.005583 (2018).

[2] Zhao, H., Shi, J., Qi, X., Wang, X. & Jia, J. in 2017 IEEE Conference on Computer Vision and Pattern Recognition (CVPR). 6230-6239.

[3] Zunair, H., Rahman, A., Mohammed, N. & Cohen, J. P. in PRIME@MICCAI.

[4] Koelstra, S., Pantic, M. & Patras, I. A Dynamic Texture-Based Approach to Recognition of Facial Actions and Their Temporal Models. IEEE Transactions on Pattern Analysis and Machine Intelligence 32, 1940-1954, doi: 10.1109/TPAMI.2010.50 (2010).

[5] Tian, Y., Kanade, T. & Cohn, J. Facial Expression Recognition. In: Li S., Jain A. (eds) Handbook of Face Recognition. Springer, London. https://doi.org/10.1007/978-0-85729-932-1_19 (2011).

[6] Hammal, Z., Kunz, M., Arguin, M. & Gosselin, F. in Proceedings of the 2008 international conference on Visions of Computer Science: BCS International Academic Conference 191-210 (BCS Learning &amp; Development Ltd., London, U K, 2008).

[7] Werner, P., Al-Hamadi, A., Limbrecht-Ecklundt, K., Walter, S., Gruss, S. & Traue, H. C. Automatic Pain Assessment with Facial Activity Descriptors. IEEE Transactions on Affective Computing 8, 286-299, doi: 10.1109/TAFFC.2016.2537327 (2017).

[8] U.S. Patent Publication No. 20220409041.

1. A method to measure of visual photosensitivity discomfort threshold (e.g., objective visual photosensitivity discomfort threshold or presence of a condition associated therewith within a subject, the method comprising:
obtaining, by one or more processors, a plurality of images of the subject, wherein each image includes a representation of i) at least one pupil and ii) a corresponding palpebral fissure contour of the subject captured while the at least one pupil and corresponding palpebral fissure contour was being subjected to a light stimulus;
determining, by the one or more processors, executing instructions for a neural network having been configured using a plurality of images of a plurality of subjects captured at a plurality of different illumination levels, an output value corresponding to a measure of visual photosensitivity discomfort threshold in which the visual photosensitivity discomfort threshold is defined by an estimated illuminance of a retina of the subject, and
outputting, by the one or more processors, the output value in a report for a stimulus illumination tool for VPT/OVPT measurement,
wherein the output value is used to diagnose the subject for photophobia or light sensitivity condition; or used to evaluate an effectiveness or adjust a configuration of eyewear or optical instruments for reducing a subject's reaction or discomfort to light sensitivity-causing stimuli.

2. The method of claim 1 further comprising:
outputting, by the one or more processors, visualization indicating an estimated area of the at least one pupil in at least one image, or a portion thereof, of the plurality of images of the subject, wherein the estimated area was generated by the neural network and used by the neural network to determine the output value.

3. The method of claim 1 further comprising:
outputting, by the one or more processors, visualization indicating a palpebral fissure contour in at least one image, or a portion thereof, of the plurality of images of the subject, wherein the palpebral fissure contour was generated by the neural network and used by the neural network to determine the output value.

4. The method of claim 1, wherein the plurality of images are acquired from an ocular photosensitivity analyzer.

5. The method of claim 1, wherein the plurality of images are acquired from a video camera system comprising illumination control.

6. The method of claim 1, wherein the neural network had been trained with a second plurality of images of a second plurality of subjects quantified as having different eye sizes.

7. The method of claim 1, wherein the neural network was trained with a second plurality of images of the plurality of subjects at different head motions.

8. The method of claim 1, wherein the obtained plurality of images each includes a representation of a facial area of the subject, wherein the neural network has been configured to classify facial expressions, and changes thereof, selected from the group consisting of blinking rate, squinting, and frowning, and wherein parameters associated with the classified facial expressions are used to generate the determined output value.

9. The method of claim 1, wherein the neural network employs feature extracted from at least one of: (i) a developed eye model, (ii) an optical model of an ocular photosensitivity analyzer or camera system, or (iii) a physical eye model comprising a variable associated with pupil diameter and/or refractive error.

10. The method of claim 1, wherein the obtained plurality of images each includes a representation of a facial area of the subject, wherein a second neural network has been configured to generate a facial expression parameter, and wherein the facial expression parameter is used to generate the determined output value.

11. The method of claim 1, wherein the neural network employs a feature extracted from a physical facial model of the subject.

12. The method of claim 1, wherein the neural network comprises an image segmentation algorithm.

13. The method of claim 1, wherein the output value defined by the estimated illuminance of the retina of the subject and as generated by the neural network, has a greater measurement repeatability as compared to the output value being defined by stimuli illuminance.

14. The method of claim 1, wherein the output value is used to diagnose the subject for photophobia or light sensitivity condition.

15. The method of claim 1, wherein the output value is used to evaluate the effectiveness of eyewear or optical instruments.

16. The method of claim 1, wherein the output value is used to adjust the configuration of eyewear or optical instruments for reducing a subject's reaction or discomfort to the light sensitivity-causing stimuli.

17. The method of claim 1, wherein the one or more processors are located in a local computing device or a cloud platform.

18. A system comprising:
a processor;
a memory having instructions stored thereon, wherein the instructions, when executed by the processor, cause the processor to:
measure of visual photosensitivity discomfort threshold or presence of a condition associated therewith within a subject by:
obtaining, a plurality of images of the subject, wherein each image includes a representation of i) at least one pupil and ii) a corresponding palpebral fissure contour, of the subject captured while the at least one pupil and corresponding palpebral fissure contour was being subjected to a light stimuli;
determining executing instructions for a neural network having been configured (trained) using a plurality of images of a plurality of subjects captured at a plurality of different illumination levels, an output value corresponding to a measure of visual photosensitivity discomfort threshold in which the visual photosensitivity discomfort threshold is defined by an estimated illuminance of the retina of the subject, and
outputting the output value in a report for a stimulus illumination tool for VPT/OVPT measurement,
wherein the output value is used to diagnose the subject for photophobia or light sensitivity condition; or used to evaluate an effectiveness or adjust a configuration of eyewear or optical instruments for reducing a subject's reaction or discomfort to light sensitivity-causing stimuli.

19. The system of claim 18, further comprising an ocular photosensitivity analyzer, wherein
the ocular photosensitivity analyzer comprises:
at least one hardware processor;
a programmable light source comprising a plurality of multi-spectra light modules configured to emit light at a range of wavelengths; a sensing system comprising one or more sensors; and
one or more software modules that are configured to, when executed by the at least one hardware processor,
receive an indication of a lighting condition comprising one or more wavelengths of light,
configure the programmable light source to emit light according to the lighting condition, and, for each of one or more iterations,
activate the programmable light source to emit the light according to the lighting condition, and collect a response, by a subject, to the emitted light via the sensing system.

20. A non-transitory computer-readable medium having instructions stored thereon, wherein execution for the instructions by the processor causes the processor to:
measure of visual photosensitivity discomfort threshold or presence of a condition associated therewith within a subject by:
obtaining, a plurality of images of the subject, wherein each image includes a representation of i) at least one pupil and ii) a corresponding palpebral fissure contour, of the subject captured while the at least one pupil and corresponding palpebral fissure contour was being subjected to a light stimuli;
determining executing instructions for a neural network having been configured using a plurality of images of a plurality of subjects captured at a plurality of different illumination levels, an output value corresponding to a measure of visual photosensitivity discomfort threshold in which the visual photosensitivity discomfort threshold is defined by an estimated illuminance of the retina of the subject, and
outputting the output value in a report for a stimulus illumination tool for VPT/OVPT measurement, wherein the output value is used to diagnose the subject for photophobia or light sensitivity condition; or used to evaluate the effectiveness or adjust the configuration of eyewear or optical instruments for reducing a subject's reaction or discomfort to light sensitivity causing stimuli.
